(19) United States
(12) Patent Application Publication
(10) Pub. No.: US 2026/0078076 A1
(43) Pub. Date: Mar. 19, 2026

(54) METHOD OF MANUFACTURING A CATALYST

(71) Applicant: Technische Universität Wien, Wien (AT)

(72) Inventors: Gernot PACHOLIK, Wien (AT); Karin FÖTTINGER, Vöcklabruck (AT)

(21) Appl. No.: 19/402,628

(22) Filed: Nov. 26, 2025

Related U.S. Application Data
(62) Division of application No. 18/029,583.

(30) Foreign Application Priority Data
Oct. 8, 2020 (AT) A 50869/2020

(51) Int. Cl. C07C 29/153; B01J 21/04; B01J 27/051; B01J 37/03; B01J 37/04; B01J 37/06; B01J 37/08; B01J 37/10

(52) U.S. Cl. CPC C07C 29/153; B01J 21/04; B01J 27/051; B01J 37/035; B01J 37/04; B01J 37/06; B01J 37/088; B01J 37/10

(57) ABSTRACT

A process for the production of methanol (CH₃OH) from carbon dioxide (CO₂) and hydrogen (H₂), wherein CO₂ is reacted with H₂ over a manganese-promoted molybdenum(IV) sulfide catalyst; as well as a catalyst for such a process and a production process for the catalyst.

### CROSS-REFERENCE TO RELATED APPLICATIONS

This Application is a division of U.S. patent application Ser. No. 18/029,583, filed Mar. 30, 2023, which is a 371 of International Patent Application No. PCT/AT2021/060369, filed Oct. 8, 2021, which claims the benefit of Austria Patent Application No. 50869/2020, filed Oct. 8, 2020, which are incorporated by reference in their entirety.

### BACKGROUND OF THE INVENTION

The present invention relates to a process for the catalytic production of methanol from carbon dioxide and hydrogen. Furthermore, the invention relates to a catalyst for the production of methanol from carbon dioxide and hydrogen. Finally, the invention relates to the use of a catalyst for the production of methanol from carbon dioxide and hydrogen.

According to the prior art, various processes are available for the fully synthetic production of alcohols. In industrial terms, processes in which carbon monoxide (CO) or carbon dioxide (CO₂) serve as starting materials are of particular importance. These processes are divided into processes for the production of methanol (CH₃OH) on the one hand and processes for the production of higher alcohols, i.e., alcohols with more than one carbon atom, on the other hand. The selectivity and the yield of the desired alcohol are important criteria.

According to the prior art, the industrial production of methanol is effected, for example, via the hydrogenation of carbon monoxide or carbon dioxide, in each case at high pressures, using suitable catalysts. Both reactions occur during the hydrogenation starting from a synthesis gas, although the yield of CO₂ hydrogenation is in need of improvement.

Liu et al., Journal of the Taiwan Institute of Chemical Engineers, 76 (2017), page 18, describe the production of higher alcohols by means of CO₂ hydrogenation with a Mo—Co—K sulfide catalyst, wherein the catalyst may comprise MoS₂, among other things.

Qi et al., Catalysis Communication, 4 (2003), page 339, describe CO hydrogenation by means of K/MoS₂. The addition of manganese to the catalyst is described as well, with a Ni/Mn/K/MoS₂ catalyst finally being described as suitable for the hydrogenation of CO. The result shows a very high selectivity for alcohols with an overall yield of 81.7%. Therein, methanol accounts for 45.8% and higher alcohols account for 53.3% (Cn alcohols with n=2, 3, 4 and 5).

Zeng et al., Applied Catalysis B: Environmental, 246 (2019), page 232, describe the production of higher alcohols with at least three carbon atoms (Cn alcohols with n≥3) by means of CO hydrogenation over MoS₂ promoted with potassium.

The selective processes known from the prior art for the production of methanol are often based on carbon monoxide as the starting material. Processes based on CO₂ either are not very selective or require expensive catalysts or, respectively, complex process conditions. If CO is to be used as a starting material, an additional reaction step is necessary for the production of CO, whereas CO₂ is available in practically unlimited quantities (for example, it accumulates as a waste product in the form of flue gas during the combustion of hydrocarbons).

### SUMMARY

It is the object of the present invention to provide a selective and inexpensive process and a catalyst for the selective hydrogenation of CO₂ to methanol. Furthermore, the catalyst should be sulfur tolerant, i.e., tolerant of trace amounts of sulfur compounds in the reaction gas.

This object is achieved by a selective process for the production of methanol (CH₃OH, MeOH) from carbon dioxide (CO₂) and hydrogen (H₂), wherein CO₂ is reacted with H₂ over a manganese-promoted MoS₂ catalyst.

Further advantages and details of the invention are shown in the accompanying figures and are explained in further detail in the following description.

### BRIEF DESCRIPTION OF THE DRAWINGS

FIG. 1 shows the reaction yield of methanol from the reaction of CO₂ with H₂ over a MoS₂ catalyst promoted with manganese as a function of temperature in a process according to the invention.

FIG. 2 shows the yield of the reaction of CO with H₂ over a MoS₂ catalyst promoted with manganese as a function of temperature.

FIG. 3 shows the reaction yield of methanol from the reaction of CO₂ with H₂ as a function of temperature in a process according to the invention over a MoS₂ catalyst promoted with manganese without potassium (▪) and with potassium (●).

FIG. 4 shows a comparison of the reaction yields of methanol or, respectively, CH₄ from the reaction of CO₂ with H₂ as a function of temperature in a process according to the invention over a MoS₂ catalyst promoted with manganese without potassium (▪) and with potassium (●).

FIG. 5 shows the comparison of the reaction yield with a cobalt-promoted MoS₂ catalyst with potassium starting from CO₂ and H₂.

FIG. 6 shows the comparison of the reaction yield with a cobalt-promoted MoS₂ catalyst with potassium starting from CO and H₂.

FIG. 7 shows different catalysts in the reaction of CO₂ with H₂ as a function of temperature.

FIG. 8 shows a comparison of the reaction yields of methanol or, respectively, CO and CH₄ from the reaction of CO₂ with H₂ in a process according to the invention over various MoS₂ catalysts promoted with manganese with different proportions of Mn and Mo.

FIG. 9 shows a comparison of the reaction yields of methanol or, respectively, CO and CH₄ from the reaction of CO₂ with H₂ over a Mn (0.30) MoS₂ catalyst in the presence of 20% helium (on the left) and without helium (on the right).

FIG. 10 shows a comparison of the reaction yields of methanol or, respectively, CO and CH₄ from the reaction of CO₂ with H₂ over a Mn (0.25) MoS₂ catalyst without a carrier (on the left), a MoS₂ catalyst with an AlO(OH) carrier (in the middle) and a Mn (0.25) MoS₂ catalyst with an AlO(OH) carrier (on the right).

FIG. 11 shows a comparison of the reaction yields of methanol from the reaction of CO₂ with H₂ as a function of temperature over a MnMoS₂ catalyst and a cobalt-promoted MoS₂ catalyst.

FIG. 12 shows a comparison of the reaction yields of methanol from the reaction of CO with H₂ as a function of temperature over a MnMoS₂ catalyst and a cobalt-promoted MoS₂ catalyst.

### DETAILED DESCRIPTION OF THE INVENTION

The invention is based on the finding that a manganese-promoted MoS₂ catalyst catalyzes the CO₂ hydrogenation in a highly selective manner. In particular, the very high yield of methanol and the high specificity of the formation of methanol are surprising in comparison to already known sulfur-tolerant catalysts. In this process, there is almost no formation of higher alcohols, and the amount of by-products formed, such as CO or CH₄, is also small.

Although the exact reaction mechanism is not yet known to the inventors, a two-stage reaction sequence with an upstream RWGS step (Reverse Water-Gas Shift Reaction) and a subsequent CO hydrogenation to methanol

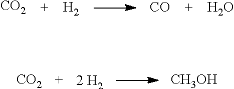

is rather improbable, since comparative tests on the MoS₂ catalyst promoted with manganese have shown hardly any conversion of CO with H₂ to form CH₃OH. The high yield and selectivity is all the more surprising.

Therefore, in one aspect, the invention relates to the use of a manganese-promoted MoS₂ catalyst for the production of methanol from CO₂ and H₂.

The object stated at the outset is further achieved by a catalyst comprising manganese-promoted molybdenum (IV) sulfide (MoS₂), the manganese-promoted molybdenum (IV) sulfide having a layered structure which can have various disorders. The structure can be described by way of the borderline cases 2H—MoS₂ and 3R—MoS₂. The proportion of manganese is such that the molar ratio of Mn to Mo is 0.1 to 0.5:1, preferably 0.2 to 0.4:1. Furthermore, XPS studies have shown that manganese can exist in the oxidation stages (II) and (III).

Manganese can preferably be present as Mn(II) sulfide and/or Mn(III) sulfide. In addition, manganese can also be present as Mn(II) oxide, Mn(III) oxide, Mn(II) hydroxide, Mn(III) hydroxide or MnOOH.

According to the invention, the manganese-promoted molybdenum (IV) sulfide (MoS₂) can be a mixed crystal of manganese sulfide(s) and MoS₂, with the basic structure being formed by the MoS₂ and manganese sulfide(s) being incorporated into this basic structure, wherein, optionally, manganese oxide(s), manganese hydroxide(s) and/or MnOOH are additionally incorporated into the basic structure according to the previous paragraph.

Additionally, the catalyst can be promoted with potassium. In this case, a phase of a K(I) salt, preferably K₂CO₃, is present on the surface of the manganese-promoted molybdenum (IV) sulfide. Such a catalyst is hereinafter referred to as manganese-promoted molybdenum (IV) sulfide with potassium.

The catalyst can additionally have a carrier on which the manganese-promoted molybdenum (IV) sulfide (optionally with potassium) is applied. The carrier can be a porous material. For example, the carrier can be an aluminium oxide or aluminium oxide hydroxide such as Al₂O₃ or AlO(OH).

The catalyst preferably consists of Mn (0.1 to 0.50) MoS₂, preferably Mn (0.2 to 0.4) MoS₂, optionally with a carrier as mentioned above.

The catalyst described above has proved to be useful for the process. Therefore, in one aspect, the invention relates to such a catalyst.

Furthermore, reaction conditions in the process have proved to be advantageous which have a pressure that has been increased in comparison to standard conditions. Therefore, it is preferably intended for the reaction to take place at a pressure of ≥10 bar. For example, the pressure can be 10 bar to 200 bar or 10 bar to 100 bar. In one embodiment variant, the pressure was between 18 bar and 23 bar.

In principle, the reaction can proceed over a wide temperature range. Suitable temperatures are, for example, between 140° C. and 320° C. If a pure manganese-promoted MoS₂ catalyst is used, the ideal temperature range is preferably between 170° C. and 220° C.

If a manganese-promoted MoS₂ catalyst mixed with potassium is used, the ideal temperature range for the reaction is somewhat higher, namely preferably between 260° C. and 300° C.

It is preferably envisaged that the partial pressure ratio of CO₂ to H₂ is about 1:2.5 to 3.5, preferably approximately 3. This means that the partial pressure of hydrogen should be about 2.5 to 3.5 times higher than the partial pressure of CO₂.

Furthermore, it has surprisingly been shown that the addition of an inert gas to the reaction mixture of CO₂ and H₂, for example, of a noble gas (such as, e.g., helium) or of nitrogen, hardly impedes the reaction. The yield decreased only slightly. The partial pressure of inert gas can be about 1:0.5 to 1.5-based on CO₂. The realization that inert gas does not interfere with the reaction means that flue gas, which contains mostly nitrogen, can also be used as a source of CO₂.

In one embodiment variant, the CO₂ can therefore come from flue gas. In this case, the process according to the invention is a selective process for the production of methanol from CO₂ and H₂, the source of CO₂ being flue gas, wherein CO₂ is reacted with H₂ over a manganese-promoted MoS₂ catalyst. Such a process is suitable for subjecting flue gas to recycling.

Although all manganese-promoted MoS₂ catalysts are suitable as catalysts, those presented according to the next-described process prove to be particularly efficient. Therefore, in one aspect, the invention relates to a process for the production of a manganese-promoted MoS₂ catalyst for the production of methanol from CO₂ and H₂, comprising the steps of:
a. forming a mixture of water, ammonium molybdate (particularly (NH₄)₆Mo₇O₂₄·4H₂O), thiourea (CH₄N₂S) and a water-soluble manganese (II) salt;
b. raising the temperature of this mixture in an autoclave to 150-250° C. and increasing the pressure to such a level that part of the water remains liquid, maintaining the temperature and pressure until the thiourea decomposes and a sulfide mixture comprising manganese sulfide and MoS₂ forms;
c. washing the sulfide mixture obtained from step (ii);
d. drying the washed sulfide mixture from step (iii);
e. calcining the dried and washed sulfide mixture from step (iv) under inert gas to obtain the manganese-promoted MoS₂ catalyst.

The sulfide mixture in step (ii) and in subsequent steps may comprise Mn(II) oxide, Mn(III) oxide, Mn(II) hydroxide, Mn(III) hydroxide or MnOOH. In addition, these compounds form, in particular, at the upper end of the temperature range.

The pressure in the autoclave preferably ranges from 5 to 40 bar, preferably it is about 15.5 bar.

Optionally, potassium can also be added to the washed sulfide mixture before it is calcined, but after it has been dried. The addition of potassium can take place in the form of an aqueous K(I) solution, e.g., a K₂CO₃ solution, wherein a drying step is then provided before the calcination. The K(I) solution can be added via ultrasonic dispersion.

Furthermore, a carrier can be provided for the catalyst. In this case, the sulfide mixture is mixed with a carrier prior to the calcination step. The carrier can be a porous material. Aluminium oxides, e.g., AlO(OH) or Al₂O₃, have proved to be suitable carriers.

Preferably, the carrier is precipitated, preferably from a precursor compound, while raising the temperature of the mixture in the autoclave to 150-250° C. and increasing the pressure to such a level that part of the water remains liquid. The precursor can be Al(NO₃)₃, for example, and initially it is present in a dissolved state. Subsequently, the dissolved precursor can be precipitated as Al(OH)₃ or AlO(OH).

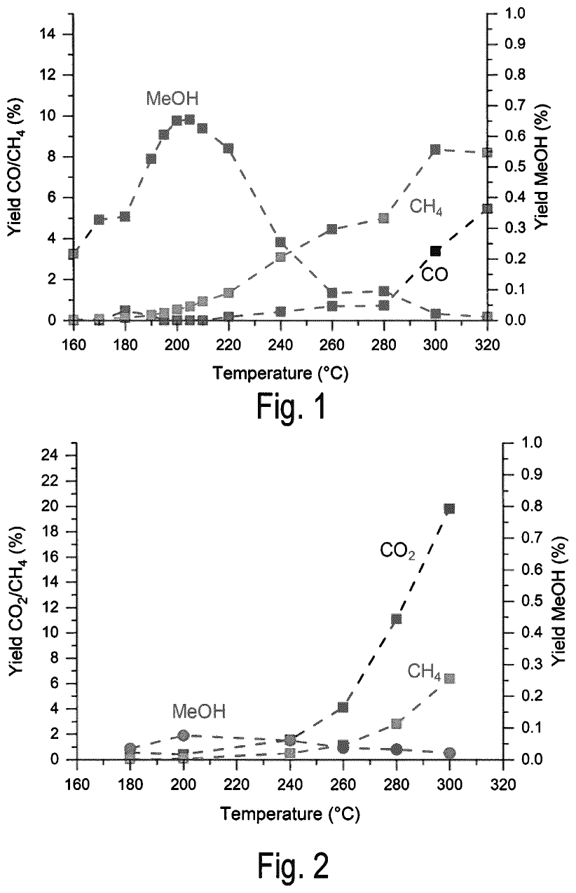

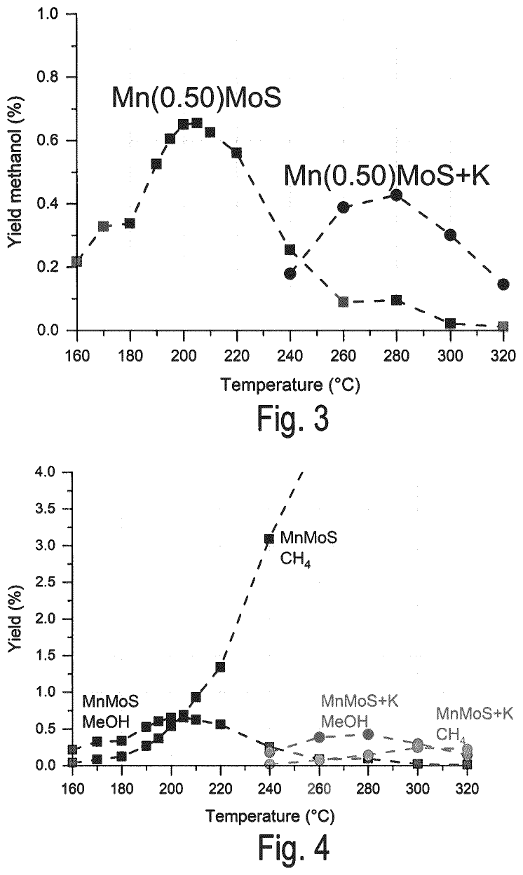

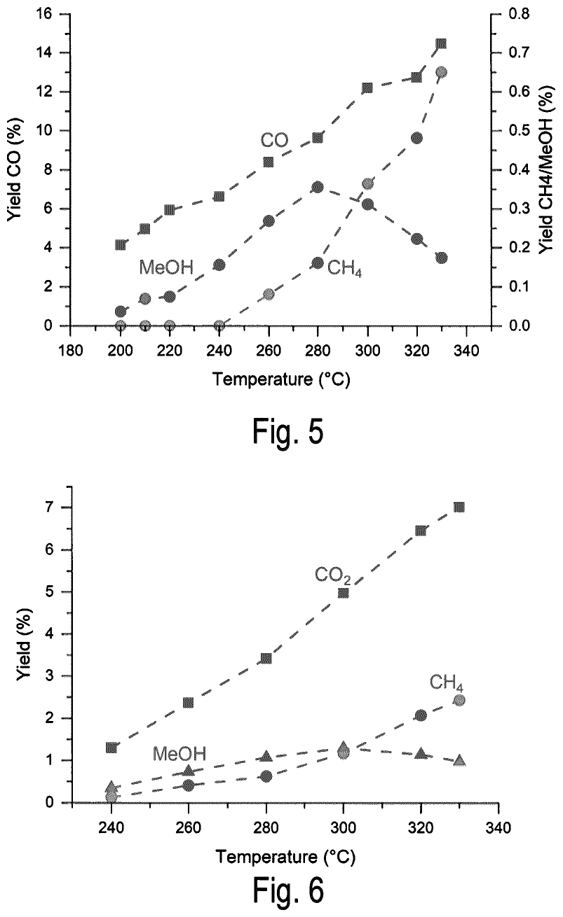

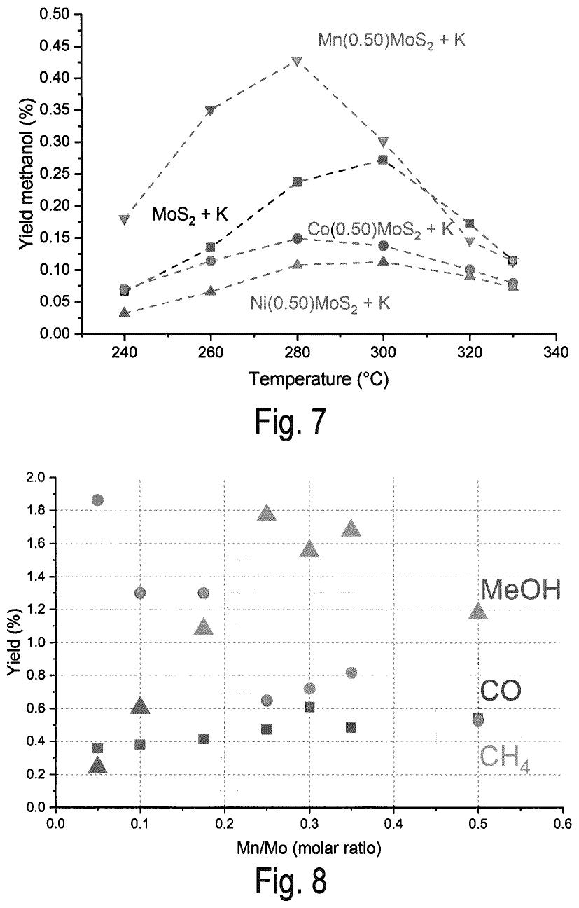

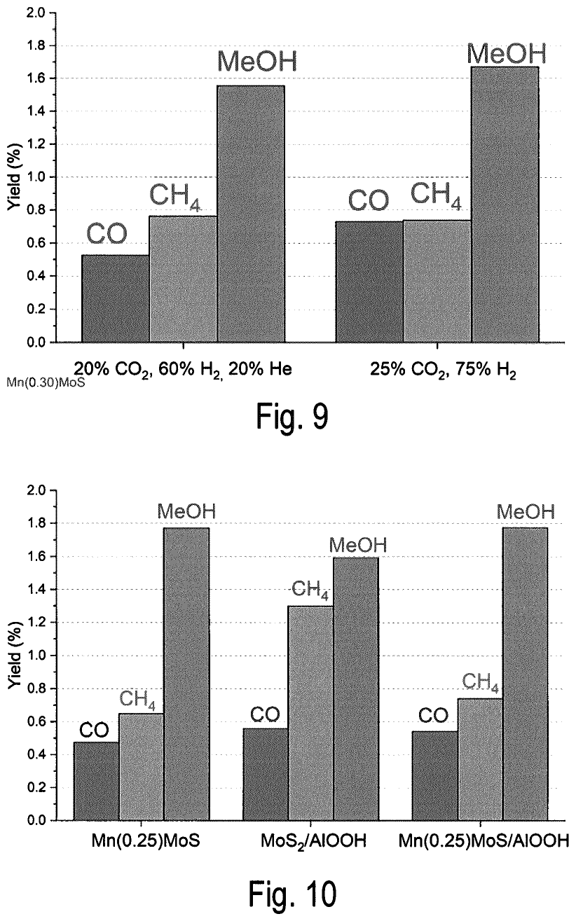

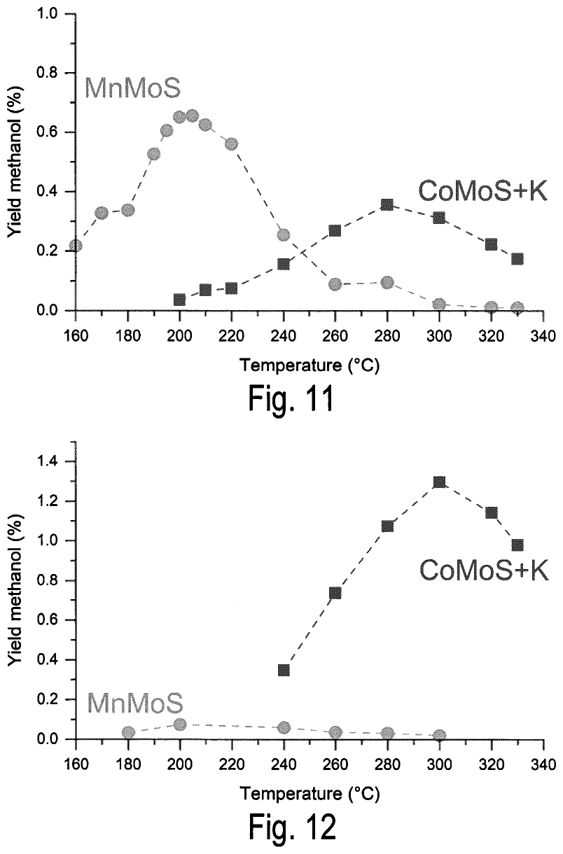

The reaction conditions in the examples shown in the figures at the beginning of the reaction, the way how the gas mixture is passed over the catalyst, are summarized in Table 1.

| | | Partial | Partial | Partial | Partial |
|---|---|---|---|---|---|
| | Total | pressure | pressure | pressure | pressure |
| FIGURE | pressure | CO₂ | CO | H₂ | He |
| FIG. 1 | 21 bar | 20% | 0 | 60% | 20% |
| FIG. 2 | 21 bar | 0% | 20% | 60% | 20% |
| FIG. 3 | 21 bar | 20% | 0 | 60% | 20% |
| FIG. 4 | 21 bar | 20% | 0 | 60% | 20% |
| FIG. 5 | 21 bar | 20% | 0 | 60% | 20% |
| FIG. 6 | 21 bar | 0% | 20% | 60% | 20% |
| FIG. 7 | 21 bar | 20% | 0 | 60% | 20% |
| FIG. 8 | 21 bar | 20% | 0 | 60% | 20% |
| FIG. 9 | 21 bar | 20% | 0 | 60% | 20% |
| | 21 bar | 25% | 0 | 75% | 0% |
| FIG. 10 | 21 bar | 20% | 0 | 60% | 20% |
| FIG. 11 | 21 bar | 20% | 0 | 60% | 20% |
| FIG. 12 | 21 bar | 20% | 0 | 60% | 20% |

The total flow of the gas mixture as it is passed over the catalyst is:

$$\frac{300 ml N}{gkat * h}$$

In this formula, “ml N” stands for millilitres under normal or standard conditions, i.e., at 273.15 K or 0° C. and 1 bar pressure. The normalization to normal conditions is carried out because, under 21 bar, 1 ml would have a higher molar number than under 1 bar; therefore, the flow is converted and related to the volume flow under normal conditions.

In FIG. 1, the reaction yield of methanol as a function of temperature in a process according to the invention is shown, when CO₂ is allowed to react with H₂ over a simple molybdenum (IV) sulfide catalyst promoted with manganese. It can be seen very clearly that there is a maximum yield of methanol at around 200° C. to 210° C., while only a few by-products are formed at this temperature. With rising temperature, the formation of methane (CH₄) increases, while the yield of methanol decreases. The amount of carbon monoxide (CO) formed also increases with rising temperature. The ideal temperature range is therefore around 180° C. to 220° C.

FIG. 2 shows, in comparison to the example of FIG. 1, that the reaction yield of methanol as a function of temperature is extremely low in a process in which CO is allowed to react with H₂ over a molybdenum (IV) sulfide catalyst promoted with manganese. As the temperature rises, the formation of CO₂ and CH₄ begins. CO should therefore be irrelevant during the formation of methanol on said catalyst.

In FIG. 3, the reaction yields of the reaction CO₂+2 H₂→CH₃OH over a simple MoS₂ catalyst promoted with manganese (▪; see example of FIG. 1) and over a MoS₂ catalyst promoted with manganese further with potassium (●) as a function of temperature in the process according to the invention are compared. As already described in FIG. 1, in case of simple manganese-promoted molybdenum (IV) sulfide, the reaction processes show a maximum yield at around 200 to 210° C. In case of the manganese-promoted MoS₂ catalyst with potassium, the maximum yield shifts to around 280° C. The addition of potassium therefore shifts the maximum yield towards higher temperatures, while a reduction in the yield (mol % based on the CO₂ used) from just under 0.7% to approx. 0.4% can be observed at the same time. However, the disadvantage resulting from the use of the manganese-promoted MoS₂ catalyst with potassium in the form of a lower yield combined with a higher ideal temperature range is accompanied by the advantage of a significant decrease in the formation of CH₄, with CH₄ being an undesirable by-product. This correlation is also illustrated in FIG. 4, where it is evident in this chart that, in case of simple (i.e., potassium-free) manganese-promoted molybdenum (IV) sulfide, the maximum yield of CH₃OH is already associated with a significant increase in the yield of CH₄ at approx. 200 to 210° C. In case of the manganese-promoted MoS₂ catalyst with potassium, the methane yield is still low at the maximum yield for CH₃OH at 280° C.

FIG. 5 shows the reaction yield of the reaction CO₂+2H₂→CH₃OH as a function of temperature in a process over a MoS₂ catalyst promoted with cobalt. The maximum yield occurs at around 280° C. It is not difficult to see that, in comparison to MoS₂ catalysts promoted with manganese (with and without potassium), not only is the amount of CH₄ formed comparatively high, but especially also the amount of CO formed is so high that this catalyst is mostly unselective for the formation of methanol. The yield of CO is higher by orders of magnitude than that of methanol already at approx. 200° C., and the yield of CH₄ also increases significantly from around 280° C.

FIG. 6 shows, in comparison to the example of FIG. 5, the reaction yield of methanol as a function of temperature in a process in which CO reacts with H₂ over a cobalt-promoted MoS₂ catalyst with potassium. The yields of methanol and methane are slightly higher overall, but CO₂ is the main product even at low temperatures and from about 300° C. the CH₄ yield exceeds the amount of CH₃OH formed.

FIG. 7 shows a comparison of the yield of methanol formed in the reaction of CO₂ with H₂ over various catalysts. A nickel-promoted MoS₂ catalyst with potassium (▴) provides the lowest yields. A MoS₂ catalyst promoted with cobalt shows only a slightly higher methanol yield (●). A MoS₂ catalyst with K (▪) shows significantly better yields, but the highest yields can be found in the process according to the invention with manganese-promoted MoS₂ with potassium (▾).

The chart of FIG. 8 shows the comparison of the reaction yields of methanol (MeOH) or, respectively, CO and CH₄ from the reaction of CO₂ with H₂ in a process according to the invention over various manganese-promoted MoS₂ catalysts with different proportions of Mn and Mo. The abscissa shows the molar proportion of manganese in relation to molybdenum. The maximum methanol yield is from 0.2 to 0.4. (Reaction conditions: 21 bar, 180° C., 20% CO₂, 60% H₂, 20% He, 300 mlN/(g_{catalyst}*h)

The column chart of FIG. 9 illustrates the reaction yields of methanol, CH₄ and CO from the reaction of CO₂ with H₂ over a Mn (0.30) MoS₂ catalyst in the presence and absence of helium as an inert gas. The yields of a mixture of 20% CO₂, 60% H₂ and 20% He are illustrated in the left-hand chart, a mixture of 25% CO₂ and 75% H₂ is illustrated in the right-hand chart. It can be seen that the yield of methanol decreases only slightly in the presence of He, surprisingly, the yield of CO decreases at the same time by a significant amount. (The reaction conditions are in each case 21 bar, 180° C., 300 mlN/(g_{catalyst}*h)).

The column chart of FIG. 10 illustrates the reaction yields of methanol, CH₄ and CO from the reaction of CO₂ with H₂ over three different catalysts. The left-hand chart shows the yields over a manganese-promoted MoS₂ catalyst (Mn (0.25) MoS₂), the middle chart shows yields on a “simple” MoS₂ catalyst, and the right-hand chart shows those on a manganese-promoted MoS₂ catalyst (Mn (0.25) MoS₂) applied to an AlO(OH) carrier. A significantly higher selectivity of the two manganese-promoted MoS₂ catalysts with regard to methanol can be seen, the significantly lower yields of the undesired by-product CH₄ are particularly striking (reaction conditions: in each case 21 bar, 180° C., 20% CO₂ 60% H₂ 20%, He 300 mlN/(g_{catalyst}*h))

FIG. 11 shows the comparison of the reaction yields of methanol of a manganese-promoted MoS₂ catalyst and a cobalt-promoted MoS₂ catalyst with potassium from the reaction of CO₂ with H₂ as a function of temperature. The reaction yields with the manganese-promoted MoS₂ catalyst are not only higher, but also shifted toward lower temperatures. (Reaction conditions: in each case 21 bar, 180° C., 20% CO₂ 60% H₂ 20% He, 300 mlN/(g_{catalyst}*h).

Furthermore, FIG. 12 shows the comparison of the reaction yields of methanol of a manganese-promoted MoS₂ catalyst and a cobalt-promoted MoS₂ catalyst with potassium from the reaction of CO with H₂ as a function of temperature. In this depiction, the selectivity of the manganese-promoted MoS₂ catalyst can be seen even more clearly. (Reaction conditions: in each case 21 bar, 180° C., 20% CO 60% H₂ 20% He, 300 mlN/(g_{catalyst}*h).) Since flue gas can comprise residual amounts of CO, the high selectivity which occurs when flue gas is used as a source for CO₂ constitutes an advantage over other catalysts.

In contrast to the prior art of sulfur-insensitive catalysts, the selective formation of methanol by means of CO₂ hydrogenation on a manganese-promoted MoS₂ (with or without potassium) catalyst is therefore significantly greater.

1. A method for preparing a manganese-promoted molybdenum (IV) sulfide catalyst, comprising:
(i) forming a mixture of water, ammonium molybdate ((NH₄)₆Mo₇O₂₄·H₂O), thiourea (CH₄N₂S) and a water soluble manganese (II) salt in a desired molar ratio;
(ii) raising the temperature of the mixture from step (i) in an autoclave to 150° C. to 250° C. and increasing the pressure to such a level that part of the water remains liquid, the temperature and pressure being maintained until the thiourea decomposes;
(iii) washing the mixture from step (ii);
(iv) drying the washed mixture from step (iii);
(v) calcining the dried and washed mixture from step (iv) under inert gas to obtain the manganese-promoted MoS₂ catalyst.

2. The method according to claim 1, wherein prior to step (v), the mixture is mixed with a carrier.

3. The method according to claim 2, wherein the carrier is precipitated from a precursor compound during step (ii).

4. The method according to claim 2, wherein the carrier for the catalyst comprises an aluminum oxide.

5. The method according to claim 1, wherein the sulfide mixture in step (ii) and in subsequent steps comprises Mn(II) oxide, Mn(III) oxide, Mn(II) hydroxide, Mn(III) hydroxide, MnOOH, or a combination thereof.

6. The method according to claim 1, wherein the pressure in the autoclave is between 5 and 40 bar.

7. The method according to claim 1, wherein the pressure in the autoclave is about 15.5 bar.

8. The method according to claim 1, wherein potassium is added after step (iii) and before step (v).

9. The method according to claim 8, wherein potassium is added in the form of an aqueous K(I) solution.
